(19) United States
(12) Patent Application Publication
(10) Pub. No.: US 2005/0113414 A1
(43) Pub. Date: May 26, 2005

(54) Piperidin-4-yl urea derivatives and related compounds as chemokine receptor inhibitors for the treatment of inflammatory diseases

(76) Inventors: Robert John Watson, Cambridge (GB); Johannes Wilhelm Georg Meissner, Cambrdigeshire (GB); Mark Ian Christie, Suffolk (GB); David Alan Owen, Cambridge (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/505,234

(22) PCT Filed: Feb. 19, 2003
(86) PCT No.: PCT/GB03/00720

(30) Foreign Application Priority Data
Feb. 20, 2002 (GB) 0203994.9

(51) Int. Cl. A61K 31/454; C07D 41/02; C07D 211/06; A61K 31/445

(52) U.S. Cl. 514/317; 514/319; 514/326; 546/207; 546/224; 546/205

(57) ABSTRACT

Cyclic amino derivatives of formula (1) are described: (1) wherein: m and n, which may be the same or different, is each zero or the integer 1 or 2; Alk³ is a covalent bond or a straight or branched C₁₋₆ alkylene chain; R¹ and R², which may be the same or different, is each a hydrogen atom or a straight or branched C₁₋₆ alkyl group; D is an optionally substituted aromatic or heteroaromatic group; E is an optionally substituted C₇₋₁₀ cycloalkyl, C₇₋₁₀ cycloalkenyl or C₇₋₁₀ polycycloaliphatic group; and the salts, solvates, hydrates, tautomers or N-oxides thereof. The compounds are potent and selective modulators of the interaction between CXCR3 and its chemokine ligands and are thus of use in medicine, for example in the prevention or treatment of conditions involving inappropriate T-cell trafficking such as certain inflammatory, autoimmune and immunoregulatory disorders as described hereinafter.

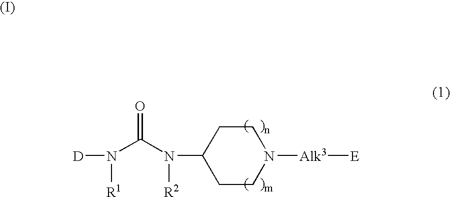

This invention relates to a series of cyclic amino derivatives, to compositions containing them, to processes for their preparation, and to their use in medicine.

Over the last few years it has become increasingly clear that chemokines (chemotactic cytokines) play a key role in the recruitment and activation of a variety of cell types in inflammatory processes, for example recruitment of eosinophils in the tissue eosinophilia that is a feature of a number of pathological conditions including asthma, rhinitis, eczema and parasitic infections. Further certain chemokines have been implicated in a variety of autoimmune diseases, such as rheumatoid arthritis, irritable bowel disease and multiple sclerosis as well as playing a critical role in the pathway of viral infection, such as invasion by HIV. [Schwarz, M. K. and Wells, T. N. C., Curr. Opin. Chem. Biol., 1999, 3, 407-17; Bousquet, J. et al, N. Eng. J. Med., 1990, 323, 1033-39; Kay, A. B. and Corrigan, C. J., Br. Med. Bull., 1992, 48, 51-64].

Chemokines are released by a wide variety of cells to attract and activate, among other cell types, macrophages, T and B lymphocytes, eosinophils, basophils and neutrophils [Luster, New Eng. J. Med., 1998, 338, 436-45; Rollins, Blood, 1997, 90, 909-28]. To date almost 40 human chemokines have been well characterised [Schwarz, M. K., ibid; Wells, T. N. C. et al, Trends Pharmacol Sci, 1998, 19, 376-380] and they have been classified into two major classes, CXC and CC, depending on whether the first two cysteines in the amino acid sequence are separated by a single amino acid (CXC) or are adjacent (CC). Members of two additional classes, C chemokines (lymphotactin-1 and lymphotactin-2) and a CX3C chemokine (fractalkine) have also been identified. It was initially thought that CXC chemokines, such as IL-8 (a neutrophil attractant), were associated with acute inflammation whilst CC chemokines were associated with chronic inflammatory diseases such as asthma, arthritis and atherosclerosis. However it is now known that members of both classes are involved in both chronic and acute inflammation.

In general the CXC chemokines, such as interleukin-8 (IL-8), neutrophil-activating protein-2 (NAP-2) and melanoma growth stimulatory activating protein (MGSA) are chemotactic primarily for neutrophils and T lymphocytes, whereas CC chemokines such as RANTES (regulation-upon-activation, normal T-cell expressed and secreted), MIP-1α, MIP-1β, the monocyte chemotactic proteins (MCP-1, MCP-2, MCP-3, MCP-4, MCP-5) and the eotaxins (-1,-2 and -3) are chemotactic for macrophages, T lymphocytes, eosinophils, dendritic cells and basophils.

The chemokines bind to specific cell-surface receptors. Seventeen mammalian receptors have been reported to date [Schwarz, M. K. ibid], all of which are seven-transmembrane-spanning G-protein coupled receptors. The ligand binding characteristics of these receptors has been identified, for example the ligands for CCR-1 are RANTES, MIP-1α and MCP-3 whilst those for CCR-2 are MCP-1, 2, 3, 4 and 5.

Chemokines and their receptors have been implicated as important mediators of inflammatory, infectious, and immunoregulatory diseases, as well as autoimmune pathologies such as rheumatoid arthritis and atherosclerosis.

The CXCR3 chemokine receptor is expressed primarily in T lymphocytes, and its functional activity can be measured by cytocolic calcium elevation or chemotaxis. The receptor was previously referred to as GPR9 or CKR-L2. Its chromosomal location is unusual among the chemokine receptors in being localised to Xq13. Ligands that have been identified that are selective and are of high affinity are the CXC chemokines, interferon-gamma inducible protein (IP10), monokine induced by interferon-gamma (MIG) and interferon-inducible T cell alpha chemoattractant (ITAC).

The highly selective expression of CXCR3 makes it an ideal target for the intervention to interrupt inappropriate T cell trafficking. The clinical indications for such intervention are in T-cell mediated diseases such as multiple sclerosis, rheumatoid arthritis and type I diabetes. Inappropriate T-cell infiltration also occurs in psoriasis and other pathogenic skin inflammation conditions, although the diseases may not be true autoimmune disorders. In this regard, up-regulation of IP-10 expression in keratinocytes is a common feature in cutaneous immunopathologies. Inhibition of CXCR3 can be beneficial in reducing rejection in organ transplantation. Ectopic expression of CXCR3 in certain tumours, especially subsets of B-cell malignancies indicate that selective inhibitors of CXCR3 will have value in tumour immunotherapy, particularly attenuation of metastasis. [See, for example, Qin S. et al, J. Clin. Invest, 1998, 101, 746-754; Sørenson T. L. et al, J. Clin. Invest, 1999, 103, 807-815.]

Accordingly in view of the clinical importance of CXCR3 there is a great need for new therapeutic agents that modulate CXCR3 function. We have found a class of cyclic amino derivatives that are potent and selective modulators of the interaction between CXCR3 and its chemokine ligands. Selective modulation of this interaction can be expected to have a beneficial effect and the compounds are thus of use in medicine, for example in the prevention or treatment of conditions involving inappropriate T-cell trafficking such as certain inflammatory, autoimmune and immunoregulatory disorders as described hereinafter.

International Patent Applications WO 01-14333, WO 00-76973, WO 00-76513, WO 00-76511, WO 00-76512, WO 00-76514, WO 00-76972 and European Patent specification no. 916668 all generally disclose classes of substituted piperidine derivatives for use in modulating chemokine receptor activity in general.

International Patent Application WO 02-16353 discloses a class of bicyclic heteroaromatic derivatives as inhibitors of the interaction between CCR3 and its chemokine ligands.

European Patent specification no. 625507 discloses a general class of urea derivatives for use as ACAT inhibitors.

U.S. Pat. No. 3,424,761 discloses a class of 3-ureidopyrrolidines characterised by analgetic, central nervous system and psychopharmacologic activities.

U.S. Pat. No. 6,329,395 discloses a general class of ureas for use as neuropeptide Y5 receptor antagonists.

Thus according to the first aspect of the invention we provide a compound of formula (1): 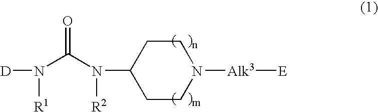
wherein:
m and n, which may be the same or different, is each zero or the integer 1 or 2;
Alk³ is a covalent bond or a straight or branched C₁₋₆ alkylene chain;
R¹ and R², which may be the same or different, is each a hydrogen atom or a straight or branched C₁₋₆ alkyl group;
D is an optionally substituted aromatic or heteroaromatic group;
E is an optionally substituted C₇₋₁₀ cycloalkyl, C₇₋₁₀ cycloalkenyl or C₇₋₁₀ polycycloaliphatic group;
and the salts, solvates, hydrates, tautomers or N-oxides thereof.

It will be appreciated that certain compounds of formula (1) may exist as geometric isomers (E or Z isomers) The compounds may also have one or more chiral centres, and exist as enantiomers or diastereomers. The invention is to be understood to extend to all such geometric isomers, enantiomers, diastereomers and mixtures thereof, including racemates. Formula (1) and the formulae hereinafter are intended to represent all individual isomers and mixtures thereof, unless stated or shown otherwise. In addition, compounds of formula (1) may exist as tautomers, for example urea (—NHC(O)NH—)—(—NC(OH)NH—) tautomers. Formula (1) and the formulae hereinafter are intended to represent all individual tautomers and mixtures thereof, unless stated otherwise.

It will also be appreciated that where desired the compounds of the invention may be administered in a pharmaceutically acceptable pro-drug form, for example, as a protected carboxylic acid derivative, e.g. as a physiologically acceptable ester. It will be further appreciated that the pro-drugs may be converted in vivo to the active compounds of formula (1), and the invention is intended to extend to such pro-drugs. Such pro-drugs are well known in the literature, see for example International Patent Application No. WO 00/23419, Bodor N. (Alfred Benson Symposium, 1982, 17, 156-177), Singh G. et al (J. Sci. Ind. Res., 1996, 55, 497-510) and Bundgaard H. (Design of Prodrugs, 1985, Elsevier, Amsterdam).

In the compounds of the invention and as represented by formula (1) and the more detailed description hereinafter certain of the general terms used in relation to substituents are to be understood to include the following atoms or groups unless specified otherwise.

Thus as used herein the term “alkyl”, whether present as a group or part of a group includes optionally substituted straight or branched C₁₋₁₀alkyl groups, for example C₁₋₆alkyl groups such as methyl, ethyl, n-propyl, i-propyl, n-butyl, i-butyl, s-butyl, t-butyl or neopentyl groups. Optional substituents when present on these groups include those optional substituents mentioned hereinafter.

The term “alkylene chain” is intended to include the alkyl groups as just described in which a terminal hydrogen atom is replaced by a covalent bond to give a divalent chain. Examples include optionally substituted C₁₋₆ alkylene chains such as —CH₂—, —CH₂CH₂—, —CH(CH₃)CH₂—, —(CH₂)₂CH₂—, —(CH₂)₃CH₂—, —CH(CH₃)(CH₂)₂CH₂—, —CH₂CH(CH₃)CH₂—, —C(CH₃)₂—, —C(CH₃)₂CH₂—, —CH₂C(CH₃)₂CH₂—, —(CH₂)₂CH(CH₃)CH₂—, —CH(CH₃)CH₂CH₂—, —CH(CH₃)CH₂CH(CH₃)CH₂—, —CH₂CH(CH₃)CH₂CH₂—, —(CH₂)₂C(CH₃)₂CH₂—, —(CH₂)₄CH₂— or —(CH₂)₅CH₂—. Optional substituents when present on these groups include those optional substituents mentioned hereinafter for alkyl groups.

In the compounds of the invention the cycloalkyl and cycloalkenyl groups represented by E include non-aromatic cyclic or multicyclic, saturated or partially saturated C₇₋₁₀ cycloalkyl or C₇₋₁₀ cycloalkenyl ring systems. Where appropriate the cycloalkyl and cycloalkenyl groups may be substituted with one or more substituents as described hereinafter.

The C₇₋₁₀ polycycloaliphatic groups represented by E include optionally substituted C₇₋₁₀bi- or tricycloalkyl or C₇₋₁₀bi- or tricycloalkenyl groups.

Examples of groups represented by E include, but are not limited to, optionally substituted cyclooctyl, cyclononyl, cyclodecyl, cyclohexenyl, cycloheptenyl, cyclooctenyl, adamantyl, adamantanonyl, noradamantyl, bicyclo[2.2.1]heptanyl, bicyclo[2.2.1]heptenyl, bicyclo[3.1.1]heptanyl, bicyclo[3.1.1]heptenyl, bicyclo[2.2.2]octanyl, bicyclo[2.2.2]octenyl, bicyclo[3.2.1]octanyl, bicyclo[3.2.1]octenyl, bicyclo[3.3.1]nonanyl, bicyclo[6.2.0]decanyl, octahydro-4,7-methanoindenyl or octahydronaphthalenyl.

Optional substituents which may be present on the group E include one, two, three or more substituents, which each may be the same or different, selected from oxo, alkoxy, haloalkyl e.g. —CF₃, —CF₂H, haloalkoxy e.g. —OCF₂H, hydroxy (—OH), thiol (—SH), alkylthio, —CN, —CO₂H, —CO₂R^{9a} (where R^{9a} is an optionally substituted C₁₋₆alkyl group), —SO₃H, —SOR^{10a} (where R^{10a} is a C₁₋₆ alkyl group) —SO₂R¹⁰, —SO₃R¹⁰, —OCO₂R¹⁰, —C(O)H, —C(O)R¹⁰, —OC(O)R¹⁰, —C(S)R¹⁰, —C(O)N(R^{11a})(R^{12a}) (where R^{11a} and R^{12a}, which may be the same or different is each a hydrogen atom or a C₁₋₆alkyl group), —N(R^{11a})C(O)R^{12a}, —CSN(R^{11a})(R^{12a}), —N(R^{11a})C(S)(R^{12a}), —SO₂N(R^{11a})(R^{12a}), —N(R^{11a})SO₂R^{12a}, —N(R^{11a})C(O)N(R^{12a})(R^{13a}) (where R^{13a} is a hydrogen atom or a C₁₋₆ alkylgroup), —N(R^{11a})C(S)N(R^{12a})(R^{13a}), N(R^{11a})SO₂N(R^{12a})(R^{13a}), or an optionally substituted cycloaliphatic, heterocycloaliphatic, aromatic or heteroaromatic group or a straight or branched C₁₋₆ alkyl or C₂₋₆ alkenyl group optionally substituted by one, two, three or more of the same or different halogen atoms, or alkoxy, haloalkyl, haloalkoxy, hydroxy (—OH), thiol (—SH), alkylthio, amino(-NH₂), substituted amino, optionally substituted C₆₋₁₂arylamino, —CN, —CO₂H, —CO₂R^{9a}, —SO₃H, —SOR^{10a}, —SO₂R¹⁰, —SO₃R¹⁰, —OCO₂R¹⁰, —C(O)H, —C(O)R¹⁰, —OC(O)R¹⁰, —C(S)R¹⁰, —C(O)N(R^{11a})(R^{12a}), —N(R^{11a})C(O)R^{12a}, —CSN(R^{11a})(R^{12a}), —N(R^{11a})C(S)(R^{12a}), —SO₂N(R^{11a})(R^{12a}), —N(R^{11a})SO₂N(R^{12a})(R^{13a}), —N(R^{11a})C(O)N(R^{12a})(R^{13a})—, —N(R^{11a})SO₂R^{12a}, —N(R^{11a})C(S)N(R^{12a})(R^{13a}), or optionally substituted cycloaliphatic, heterocycloaliphatic, aromatic or heteroaromatic groups.

In general in the compounds of formula (1) the term “cycloaliphatic group” includes optionally substituted non-aromatic cyclic or multicyclic, saturated or partially saturated C₃₋₁₀ ring systems, such as, cyclopropyl, cyclobutyl, cyclopentyl, cyclohexyl, cycloheptyl, cyclooctyl, cyclobutenyl, cyclopentenyl, cyclohexenyl, cycloheptenyl, cyclooctenyl, adamantyl, norbornyl, norbornenyl, bicyclo[2.2.1]heptanyl or bicyclo[2.2.1]heptenyl. Particular examples include optionally substituted C₃₋₆ cycloalkyl ring systems such as cyclopropyl, cyclobutyl, cyclopentyl and cyclohexyl groups. Optional substituents present on these groups include those substituents mentioned hereinafter.

The term “heterocycloaliphatic group” refers to an optionally substituted non-aromatic 3 to 10 membered saturated or partially saturated monocyclic or multicyclic hydrocarbon ring system containing one, two, three or four L³ linker atoms or groups. Particular examples of suitable L³ atoms or groups include —O— or —S— atoms or —C(O)—, —C(O)O—, —OC(O)—, —C(S)—, —S(O)—, —S(O)₂—, —N(R¹⁴)— [where R¹⁴ is a hydrogen atom or a C₁₋₆ alkyl group], —N(R¹⁴)N(R¹⁴), —N(R¹⁴)O, —ON(R¹⁴)—, —CON(R¹⁴)—, —OC(O)N(R¹⁴)—, —CSN(R¹⁴)—, —N(R¹⁴)CO—, —N(R¹⁴)C(O)O—, —N(R¹⁴)CS—, —S(O)₂N(R¹⁴)—, —N(R¹⁴)S(O)₂—, —N(R¹⁴)CON(R¹⁴)—, —N(R¹⁴)CSN(R¹⁴)—, —N(R¹⁴)SO₂N(R¹⁴)— groups. Where the linker group contains two R¹⁴ substituents, these may be the same or different. Optional substituents present on the heterocycloaliphatic groups include those substituents mentioned hereinafter.

Particular examples of heterocycloaliphatic groups include optionally substituted cyclobutanonyl, cyclopentanonyl, cyclohexanonyl, azetidinyl, tetrahydrofuranyl, tetrahydropyranyl, pyrrolinyl, e.g. 2- or 3-pyrrolinyl, pyrrolidinyl, pyrrolidinonyl, oxazolidinyl, oxazolidinonyl, dioxolanyl, e.g. 1,3-dioxolanyl, imidazolinyl, e.g. 2-imidazolinyl, imidazolidinyl, pyrazolinyl, e.g. 2-pyrazolinyl, pyrazolidinyl, thiazolinyl, thiazolidinyl, pyranyl, e.g. 2- or 4-pyranyl, pyranonyl, piperidinyl, piperidinonyl, quinuclidinyl, 1,4-dioxanyl, morpholinyl, morpholinonyl, 1,4-dithianyl, thiomorpholinyl, piperazinyl, N—C₁₋₆ alkylpiperazinyl, homopiperazinyl, dihydrofuran-2-onyl, tetrahydropyran-2-onyl, isothiazolidinyl 1,1-dioxide, [1,2]thiazinanyl 1,1-dioxide, tetrahydrothiophenyl, tetrahydrothiopyranyl, pyrazolidin-3-onyl, tetrahydrothiopyranyl 1,1-dioxide, tetrahydrothiophenyl 1,1-dioxide, 1,3,5-trithianyl, oxazinyl, e.g. 2H-1,3-, 6H-1,3-, 6H-1,2-, 2H-1,2- or 4H-1,4-oxazinyl, 1,2,5-oxathiazinyl, isoxazinyl, e.g. o- or p-isoxazinyl, oxathiazinyl, e.g. 1,2,5 or 1,2,6-oxathiazinyl, or 1,3,5,-oxadiazinyl groups.

The optional substituents which may be present on the alkyl, cycloaliphatic or heterocycloaliphatic groups described above, include one, two, three or more substituents, which each may be the same or different, selected from halogen atoms, or alkoxy, haloalkyl, haloalkoxy, hydroxy (—OH), thiol (—SH), alkylthio, amino(-NH₂), substituted amino, optionally substituted C₆₋₁₂arylamino, —CN, —CO₂H, —CO₂R⁹ (where R⁹ is an optionally substituted C₁₋₆ alkyl group), —SO₃H, —SOR¹⁰ (where R¹⁰ is a C₁₋₆ alkyl group) —SO₂R¹⁰, —SO₃R¹⁰, —OCO₂R¹⁰, —C(O)H, —C(O)R¹⁰, —OC(O)R¹⁰, —C(S)R¹⁰, —C(O)N(R¹¹)(R¹²) (where R¹¹ and R¹², which may be the same or different is each a hydrogen atom or a C₁₋₆ alkyl group), —OC(O)N(R¹¹)(R¹²), N(R¹¹)C(O)R¹², —CSN(R¹¹)(R¹²), —N(R¹¹)C(S)(R¹²), —SO₂N(R¹¹)(R¹²), —N(R¹¹)SO₂R¹², —N(R¹¹)C(O)N(R¹²)(R¹³) (where R¹³ is a hydrogen atom or a C₁₋₆ alkyl group), —N(R¹¹)C(S)N(R¹²)(R¹³), —N(R¹¹)SO₂N(R¹²)(R¹³), or optionally substituted aromatic or heteroaromatic groups or a C₁₋₆ alkyl group optionally substituted by one, two, three or more of the same or different atoms or groups selected from halogen atoms, or alkoxy, haloalkyl, haloalkoxy, hydroxy, thiol, alkylthio, amino, substituted amino, optionally substituted C₆₋₁₂arylamino, —CN, —CO₂H, —CO₂R⁹, —SO₃H, —SOR¹⁰, —SO₂R¹⁰, —SO₃R¹⁰, —OCO₂R¹⁰, —C(O)H, —C(O)R¹⁰, —OC(O)R¹⁰, —C(S)R¹⁰, —C(O)N(R¹¹)(R¹²), —OC(O)N(R¹¹)(R¹²), —N(R¹¹)C(O)R¹², —CSN(R¹¹)(R¹²), —N(R¹¹)C(S)(R¹²), —SO₂N(R¹¹)(R¹²), —N(R¹¹)SO₂R¹², —N(R¹¹)C(O)N(R¹²)(R¹³), —N(R¹¹)C(S)N(R¹²)(R¹³), —N(R¹¹)SO₂N(R¹²)(R¹³), or an optionally substituted aromatic or heteroaromatic groups. Substituted amino groups include —NHR¹⁰ and —N(R¹⁰)(R¹¹) groups.

Cycloaliphatic groups may be linked to the remainder of the compound of formula (1) by any available ring carbon atom. Heterocycloaliphatic groups may be linked to the remainder of the compound of formula (1) by any available ring carbon or, where available, ring nitrogen atom.

The term “halogen atom” is intended to include fluorine, chlorine, bromine or iodine atoms.

The term “haloalkyl” is intended to include the alkyl groups just mentioned substituted by one, two or three of the halogen atoms just described. Particular examples of such groups include —CF₃, —CCl₃, —CHF₂, —CHCl₂, —CH₂F, and —CH₂Cl groups.

The term “alkoxy” as used herein is intended to include straight or branched C₁₋₁₀alkoxy for example C₁₋₆alkoxy such as methoxy, ethoxy, n-propoxy, i-propoxy and t-butoxy. “Haloalkoxy” as used herein includes any of those alkoxy groups substituted by one, two or three halogen atoms as described above. Particular examples include —OCF₃, —OCCl₃, —OCHF₂, —OCHCl₂, —OCH₂F and —OCH₂Cl groups.

As used herein the term “alkylthio” is intended to include straight or branched C₁₋₁₀alkylthio, e.g. C₁₋₆alkylthio such as methylthio or ethylthio groups.

The terms “aromatic group” and “aryl group” are intended to include for example optionally substituted monocyclic ring C₆₋₁₂ aromatic groups, such as phenyl, or bicyclic fused ring C₆₋₁₂ aromatic groups, such as, 1- or 2-naphthyl groups.

The terms “heteroaromatic group” and “heteroaryl group” are intended to include for example optionally substituted C₁₋₉ heteroaromatic groups containing for example one, two, three or four heteroatoms selected from oxygen, sulfur or nitrogen atoms (or oxidised versions thereof). In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulfur or nitrogen atoms. Bicyclic heteroaromatic groups include for example eight- to thirteen-membered fused-ring heteroaromatic groups containing one, two or more heteroatoms selected from oxygen, sulphur or nitrogen atoms.

Each of these aromatic or heteroaromatic groups may be optionally substituted by one, two, three or more R¹⁶ atoms or groups as defined below.

Particular examples of monocyclic ring heteroaromatic groups of this type include pyrrolyl, furyl, thienyl, imidazolyl, N—C₁₋₆alkylimidazolyl, oxazolyl, isoxazolyl, thiazolyl, isothiazolyl, pyrazolyl, triazolyl, oxadiazolyl, thiadiazolyl, pyridyl, pyrimidinyl, pyridazinyl, pyrazinyl, tetrazolyl, or triazinyl.

Particular examples of bicyclic ring heteroaromatic groups of this type include benzofuryl, benzothienyl, benzotriazolyl, indolyl, indazolinyl, benzimidazolyl, imidazo[1,2-a]pyridyl, benzothiazolyl, benzoxazolyl, benzisoxazolyl, benzopyranyl, quinazolinyl, quinoxalinyl, naphthyridinyl, pyrido[3,4-b]pyridyl, pyrido[3,2-b]pyridyl, pyrido[4,3-b]-pyridyl, quinolinyl, isoquinolinyl or phthalazinyl.

Optional substituents which may be present on the aromatic or heteroaromatic groups include one, two, three or more substituents, each selected from an atom or group R¹⁶ in which R¹⁶ is —R₁₆ₐ or -Alk⁴(R^{16a})_{f}, where R^{16a} is a halogen atom, or an amino (—NH₂), substituted amino, nitro, cyano, hydroxyl (—OH), substituted hydroxyl, amidino, formyl, carboxyl (—CO₂H), esterified carboxyl, thiol (—SH), substituted thiol, —COR¹⁷ [where R¹⁷ is an -Alk⁴(R^{16a})_{f}, heterocycloaliphatic, cycloaliphatic, aryl or heteroaryl group], —CSR¹⁷, —SO₃H, —SOR¹⁷, —SO₂R¹⁷, —SO₃R¹⁷, —SO₂NH₂, —SO₂NHR¹⁷, SO₂N(R¹⁷)₂, —CONH₂, —CSNH₂, —CONHR¹⁷, —CSNHR¹⁷, —CON(R¹⁷)₂, —CSN(R¹⁷)₂, —N(R¹⁸)SO₂R¹⁷, [where R¹⁸ is a hydrogen atom or a C₁₋₆ alkyl group] —N(SO₂R¹⁷)₂, —N(R¹⁸)SO₂NH₂, —N(R¹⁸)SO₂NHR¹⁷, —N(R¹⁷)SO₂N(R¹⁸)₂, —N(R¹⁸)COR¹⁷, —N(R¹⁸)CONH₂, —N(R¹⁸)CONHR¹⁷, —N(R¹⁸)CON(R¹⁷)₂, —N(R¹⁸)CSNH₂, —N(R¹⁸)CSNHR¹⁷, —N(R¹⁸)CSN(R¹⁷)₂, —N(R¹⁸)CSR¹⁷, —N(R¹⁸)C(O)OR¹⁷, —SO₂NHet¹ [where —NHet¹ is an optionally substituted C₃₋₇heterocycloaliphatic group containing at least one N atom and optionally containing one or more other —O— or —S— atoms or —N(R¹⁸)—, —C(O)— or —C(S)— groups], —CONHet¹, —CSNHet¹, —N(R¹⁴)SO₂NHet¹, —N(R¹⁸)CONHet¹, —N(R¹⁸)CSNHet¹, —SO₂N(R¹⁸)Het² [where Het² is an optionally substituted monocyclic C₃₋₇ cycloalipatic group optionally containing one or more —O— or —S— atoms or —N(R¹⁸)—, —C(O)— or —C(S)— groups], -Het², —CON(R¹⁸)Het², —CSN(R¹⁸)Het², —N(R¹⁸)CON(R¹⁸)Het², —N(R¹⁸)CSN(R¹⁸)Het², optionally substituted aryl or heteroaryl group; Alk⁴ is a straight or branched C₁₋₆alkylene, C₂₋₆alkenylene or C₂₋₆alkynylene chain, optionally interrupted by one, two or three —O— or —S— atoms or —S(O)_{g}— [where g is an integer 1 or 2] or —N(R¹⁸)— groups; and f is zero or an integer 1, 2 or 3. It will be appreciated that when two R¹⁷ or R¹⁸ groups are present in one of the above substituents, the R¹⁷ or R¹⁸ groups may be the same or different.

When in the group -Alk⁴(R^{16a})_{f} f is an integer 1, 2 or 3, it is to be understood that the substituent or substituents R^{16a} may be present on any suitable carbon atom in -Alk⁴. Where-more than one R^{16a} substituent is present these may be the same or different and may be present on the same or different atom in -Alk⁴. Clearly, when f is zero and no substituent R^{16a} is present the chain represented by Alk⁴ becomes a corresponding group.

When R^{16a} is a substituted amino group it may be for example a group —NHR¹⁷ [where R¹⁷ is as defined above] or a group —N(R17)₂ wherein each R¹⁷ group is the same or different.

When R^{16a} is a substituted hydroxyl or substituted thiol group it may be for example a group —OR¹⁷ or a —SR¹⁷ group respectively.

Esterified carboxyl groups represented by the group R^{16a} include groups of formula —CO₂Alk⁵ wherein Alk⁵ is an optionally substituted alkyl group.

When Alk⁴ is present in or as a substituent it may be for example a methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene or 3-butynylene chain, optionally interrupted by one, two, or three —O— or —S—, atoms or —S(O)—, —S(O)₂— or —N(R¹⁵)— groups.

When —NHet¹ or -Het² forms part of a substituent R¹⁶ each may be for example an optionally substituted 2- or 3-pyrrolinyl, pyrrolidinyl, pyrazolinyl, pyrazolidinyl, piperazinyl, imidazolinyl, imidazolidinyl, morpholinyl, thiomorpholinyl, piperidinyl, oxazolidinyl or thiazolidinyl group. Additionally Het² may represent for example, an optionally substituted cyclopentyl or cyclohexyl group. Optional substituents which may be present on —NHet¹ or -Het² include those substituents described above in relation to aromatic groups.

Particularly useful atoms or groups represented by R¹⁶ include fluorine, chlorine, bromine or iodine atoms, or C₁₋₆alkyl, e.g. methyl, ethyl, n-propyl, i-propyl, n-butyl or t-butyl, optionally substituted phenyl, pyridyl, pyrimidinyl, pyrrolyl, furyl, thiazolyl, thienyl, morpholinyl, thiomorpholinyl, piperazinyl, pyrrolidinyl or piperidinyl, C₁₋₆hydroxyalkyl, e.g. hydroxymethyl or hydroxyethyl, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxyproplthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₅₋₇cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, aminoC₁₋₆alkylamino e.g. aminoethylamino, Het¹NC₁₋₆alkylamino e.g. morpholinopropylamino, C₁₋₆alkylaminoC₁₋₆alkyl, e.g. ethylaminoethyl, C₁₋₆ dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆ dialkylaminoC₁₋₆alkoxy, e.g. dimethylaminoethoxy, diethylaminoethoxy, diisopropylaminoethoxy, or dimethylaminopropoxy, hydroxyC₁₋₆alkylamino e.g. hydroxyethylamino, imido, such as phthalimido or naphthalimido, e.g. 1,8-naphthalimido, nitro, cyano, amidino, formyl[HC(O)—], carboxyl (—CO₂H), —CO₂Alk⁵ [where Alk⁵ is as defined above], C₁₋₆alkanoyl e.g. acetyl, optionally substituted benzoyl, thiol (—SH), thioC₁₋₆alkyl, e.g. thiomethyl or thioethyl, —SC(═NH)NH₂, sulphonyl (—SO₃H), —SO₃R¹⁸, C₁₋₆alkylsulphinyl e.g. methylsulphinyl, C₁₋₆alkylsulphonyl, e.g. methylsulphonyl, aminosulphonyl (—SO₂NH₂), C₁₋₆alkylaminosulphonyl, e.g. methylamino-sulphonyl or ethylaminosulphonyl, C₁₋₆dialkylaminosulphonyl, e.g. dimethyl-aminosulphonyl or diethylaminosulphonyl, optionally substituted phenylamino-sulphonyl, carboxamido (—CONH₂), C₁₋₆alkylaminocarbonyl, e.g. methylamino-carbonyl or ethylaminocarbonyl, C₁₋₆dialkylaminocarbonyl, e.g. dimethyl-aminocarbonyl or diethylaminocarbonyl, aminoC₁₋₆alkylaminocarbonyl, e.g. aminoethylaminocarbonyl, C₁₋₆dialkylaminoC₁₋₆alkylaminocarbonyl, e.g. diethylaminoethylaminocarbonyl, aminocarbonylamino, C₁₋₆alkylamino-carbonylamino, e.g. methylaminocarbonylamino or ethylaminocarbonylamino, C₁₋₆dialkylaminocarbonylamino, e.g. dimethylaminocarbonylamino or diethyl-aminocarbonylamino, C₁₋₆alkylaminocabonylC₁₋₆alkylamino, e.g. methylamino-carbonylmethylamino, aminothiocarbonylamino, C₁₋₆alkylaminothiocarbonyl-amino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, —CONHC(═NH)NH₂, C₁₋₆alkyl-sulphonylamino, e.g. methylsulphonylamino or ethylsulphonylamino, C₁₋₆dialkylsulphonylamino, e.g. dimethylsulphonylamino or diethylsulphonyl-amino, optionally substituted phenylsulphonylamino, aminosulphonylamino (—NHSO₂NH₂), C₁₋₆alkylaminosulphonylamino e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino e.g. aminoacetylamino, C₁₋₆dialkyl-aminoC₁₋₆alkanoylamino, e.g. dimethylaminoacetylamino, C₁₋₆alkanoyl-aminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino or optionally substituted benzyloxy, benzylamino, pyridylmethoxy, thiazolylmethoxy, benzyloxy-carbonylamino, benzyloxycarbonylaminoC₁₋₆alkyl e.g. benzyloxycarbonyl-aminoethyl, thiobenzyl, pyridylmethylthio or thiazolylmethylthio groups.

Where desired, two adjacent R¹⁶ substituents may be linked together to form a cyclic group such as a cyclic ether, e.g. a C₁₋₆alkylenedioxy group such as methylenedioxy or ethylenedioxy or a C₃₋₆ cycloalkyl or 3-10 membered monocylic heterocycloaliphatic group as defined herein.

It will be appreciated that where two or more R¹⁶ substituents are present, these need not necessarily be the same atoms and/or groups. In general, the substituent(s) may be present at any available ring position in the aromatic or heteroaromatic group.

When R¹⁰, R^{10a}, R¹¹, R^{11a}, R¹², R^{12a}, R¹³, R^{13a}, R¹⁴ or R¹⁸ is present as a C₁₋₆alkyl group it may be a straight or branched C₁₋₆ alkyl group e.g. a C₁₋₃ alkyl group such as methyl, ethyl or i-propyl.

Examples of optionally substituted alkyl groups present in ester groups of formulae —CO₂R⁹, —CO₂R^{9a} and —CO₂Alk⁵ include C₁₋₆ alkyl groups as herein described. Optional substituents which may be present on these alkyl groups include optionally substituted cycloaliphatic, aromatic or heteroaromatic groups as herein defined.

The presence of certain substituents in the compounds of formula (1) may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydroiodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

Alk³ in one group of compounds of formula (1) is preferably a C₁₋₃ alkylene chain, in particular —CH₂—, —CH₂CH₂—, —CH₂CH₂CH₂—, more particularly —CH₂—.

Alternatively Alk³ in another group of compounds of formula (1) is a covalent bond.

In compounds of formula (1) m and n, which may be the same or different, is each in particular zero or the integer 1. In particular m and n is each the integer 1.

R¹ and R², which may be the same or different, is each preferably a hydrogen atom or a straight or branched C₁₋₃ alkyl group, especially methyl. In one particular group of compounds of the invention R¹ and R² is each a hydrogen atom.

One group of compounds of the invention has the formula (1) wherein D is selected from optionally substituted phenyl, 1- or 2-naphthyl, pyrrolyl, furyl, thienyl, imidazolyl, N—C₁₋₆alkylimidazolyl, oxazolyl, isoxazolyl, thiazolyl, isothiazolyl, pyrazolyl, triazolyl, oxadiazolyl, thiadiazolyl, pyridyl, pyrimidinyl, pyridazinyl, pyrazinyl, tetrazolyl, triazinyl, benzofuryl, benzothienyl, benzotriazolyl, indolyl, indazolinyl, benzimidazolyl, benzothiazolyl, benzoxazolyl, benzisoxazolyl, benzopyranyl, quinazolinyl, quinoxalinyl, naphthyridinyl, pyrido[3,4-b]pyridyl, pyrido[3,2-b]pyridyl, pyrido[4,3-b]-pyridyl, quinolinyl or isoquinolinyl.

More particular D groups include optionally substituted phenyl, 1- or 2-naphthyl, pyridyl, pyrimidinyl, pyridazinyl, pyrazinyl, benzofuryl, benzothienyl, indolyl, benzimidazolyl, benzothiazolyl, benzoxazolyl, benzisoxazolyl, quinazolinyl, quinoxalinyl, naphthyridinyl, quinolinyl or isoquinolinyl. D may also in particular be an optionally substituted pyrrolyl, furyl, thienyl, imidazolyl, N—C₁₋₆alkylimidazolyl, oxazolyl, isoxazolyl, thiazolyl or isothiazolyl group.

In one group of compounds of formula (1) D is especially an optionally substituted phenyl or 2-naphthyl group. D is also especially an optionally substituted thienyl group.

Particular substituents, which may be present on the group D, are one, two, three or more atoms or groups selected from fluorine, chlorine, bromine, optionally substituted straight or branched C₁₋₃ alkyl (wherein the optional alkyl substituent is in particular an optionally substituted phenyl or monocyclic heteroaryl group, especially pyridyl, pyrimidinyl, pyrrolyl, furyl, thiazolyl or thienyl), optionally substituted phenyl, monocyclic heteroaryl, morpholinyl, thiomorpholinyl, piperazinyl, pyrrolidinyl, piperidinyl, methoxy, phenoxy, pyridyloxy, benzoyl, pyridoyl or COCH₃, OCF₃, OCF₂H, CF₃, NO₂, NH₂, NHCH₃, N(CH₃)₂, CONH₂, CONHCH₃, CON(CH₃)₂, CO₂CH₃, CO₂CH₂CH_{CH}₃, CO₂H or —CN, —SCH₃, —SCH₂CH₃, —SO₂CH₃ or two adjacent substituents are linked together to form methylenedioxy, ethylenedioxy or cyclopentyl. The monocyclic heteroaryl substituents in compounds of this type are in particular selected from pyridyl, pyrimidinyl, pyrrolyl, furyl, thiazolyl or thienyl.

More particular D substituents are selected from fluorine, chlorine, CF₃, methyl, ethyl, methoxy, OCF₂H, OCF₃ or optionally substituted phenyl, monocyclic heteroaryl, especially pyridyl, pyrimidinyl, pyrrolyl, furyl, thiazolyl or thienyl, phenoxy or pyridyloxy or —SCH₃. Especially useful D substituents include fluorine, chlorine, CF₃, methyl, ethyl, methoxy, —SCH₃ or optionally substituted phenyl or phenoxy. The optional substituents which may in particular be present on these aryl or heteroaryl groups are one, two, three or more atoms or groups selected from fluorine, chlorine, bromine, straight or branched C₁₋₃ alkyl, methoxy, OCF₃, OCF₂H, CF₃, CN, NO₂, NH₂, NHCH₃, N(CH₃)₂, CONH₂, CONHCH₃, CON(CH₃)₂, CO₂CH₃, CO₂CH₂CH₃ or CO₂H.

Particular examples of D groups include 3,4-dichlorobenzene, 3- or 4-chlorobenzene or 3- or 4-trifluoromethylbenzene. D is also in particular a group selected from 3,5-bistrifluoromethylbenzene, 3-methylsulfanylbenzene or 5-phenylthien-2-yl.

One group of compounds has the formula (1) wherein E is selected from optionally substituted cycloheptyl, cyclooctyl, cyclononyl, cyclohexenyl, cycloheptenyl, cyclooctenyl, adamantyl, bicyclo[2.2.1]heptanyl, bicyclo[2.2.1]heptenyl, bicyclo[3.1.1]heptanyl or bicyclo[3.1.1]heptenyl.

Particular substituents, which may be present on the group E, are one, two, three or more groups selected from hydroxy, or optionally substituted phenyl or monocyclic heteroaromatic, CONH₂, CONHCH₃, CON(CH₃)₂, CO₂CH₃, CO₂CH₂CH₃, CO₂H or optionally substituted straight or branched C₁₋₆ alkyl or C₂₋₆ alkenyl, wherein the optional alkyl or alkenyl substituent is in particular an optionally substituted phenyl or monocyclic heteroaromatic group. Particular examples of the optionally substituted C₁₋₆ alkyl or C₂₋₆ alkenyl groups are —CH₃, —CH₂CH₃, —CH(CH₃)₂, —(CH₂)₂CH₃, —(CH₂)₃CH₃, —CH(CH₃)CH₂CH₃, —CH₂CH(CH₃)₂, —CH₂C(CH₃)₃, —C(CH₃)₃, —(CH₂)₄CH₃, —(CH₂)₅CH₃ —CHCH₂, —CHCHCH₃, —CH₂CHCH₂, —CHCHCH₂CH₃, —CH₂CHCHCH₃, —(CH₂)₂CHCH₂ or —C(CH₂)CH₃.

One preferred group of compounds is where E is substituted with one, two, three or more methyl groups.

E in one particular group of compounds of the invention is a 1-cyclooctenyl or 6,6-dimethylbicyclo[3.1.1]hept-2-en-2-yl group. E is also especially an adamantyl or cyclooctyl group.

One particular group of optional substituents which may be present on cycloaliphatic or heterocycloaliphatic groups in compounds of formula (1), in particular on the D or E group substituents, are one, two or three groups selected from C₁₋₃ alkoxy, OCF₃, OCF₂H, CF₃, C₁₋₃ alkylthio, —CN, NHCH₃, N(CH₃)₂, CONH₂, CONHCH₃, CON(CH₃)₂, CO₂CH₃, CO₂CH₂CH₃, —CO₂C(CH₃)₃, —COCH₃, —NHCOCH₃, —N(CH₃)COCH₃, CO₂H, or optionally substituted straight or branched C₁₋₃ alkyl, wherein the optional alkyl substituent is in particular —CN, C₁₋₃ alkoxy, NHCH₃, N(CH₃)₂, CONH₂, CONHCH₃, CON(CH₃)₂, CO₂CH₃, CO₂CH₂CH₃, —CO₂C(CH₃)₃, —COCH₃, —NHCOCH₃, —N(CH₃)COCH₃ or CO₂H.

Particular aromatic or heteroaromatic substituents, which may be present on compounds of formula (1), in particular on the D or E group substituents, are one, two or three atoms or groups selected from fluorine, chlorine, bromine, straight or branched C₁₋₃ alkyl, methoxy, OCF₃, OCF₂H, CF₃, CN, No₂, NH₂, NHCH₃, N(CH₃)₂, CONH₂, CONHCH₃, CON(CH₃)₂, CO₂CH₃, CO₂CH₂CH₃ or CO₂H.

Particularly useful compounds of the invention include:
1-[1-((E)-1-Cyclooct-1-enyl)methylpiperidin-4-yl]-3-(3,4-dichlorophenyl)-urea;
1-[1-((E)-1-Cyclooct-1-enyl)methyl-piperidin-4-yl]-3-(4-trifluoromethylphenyl)-urea;
and the salts, solvates, hydrates, tautomers or N-oxides thereof.

Other particularly useful compounds include:
N-2-Naphthyl-N′-(cyclooctene-1-yl)methylpiperidine urea;
1-[-(6,6-Dimethylbicyclo[3.1.1]hept-2-en-2-ylmethyl)piperidin-4-yl]-3-naphthalen-2-yl urea;
1-[1-(6,6-Dimethylbicyclo[3.1.1]hept-2-en-2-ylmethyl)piperidin-4-yl]-1-methyl-3-(3-trifluoromethylphenyl)urea;
1-[1-(6,6-Dimethylbicyclo[3.1.1]hept-2-en-2-ylmethyl)piperidin-4-yl]-1-methyl-3-naphthalen-2-yl urea;
1-[1-(6,6-Dimethylbicyclo[3.1.1]hept-2-en-2-ylmethyl)piperidin-4-yl]-1-methyl-3-(3-methylsulfanylphenyl)urea;
1-(1-Adamantan-1-ylmethylpiperidin-4-yl)-3-(3-trifluoromethylphenyl)-urea;
1-[1-(6,6-Dimethylbicyclo[3.1.1]hept-2-en-2-ylmethyl)piperidin-4-yl]-1-ethyl-3-(3-methylsulfanylphenyl)urea;
′3-{3-[1-((1R,5S)-6,6-Dimethylbicyclo[3.1.1]hept-2-en-2-ylmethyl)-piperidin-4-yl]ureido}benzoic benzoic acid methyl ester;
′1-[1-((1R,5S)-6,6-Dimethylbicyclo[3.1.1]hept-2-en-2-ylmethyl)piperidin-4-yl]-3-(5-phenylthiophen-2-yl)urea;
′1-[1-((E)-1-Cyclooct-1-enyl)methylpiperidin-4-yl]-3-(5-phenylthiophen-2-yl)urea;
′1-(4-Chloro-3-trifluoromethylphenyl)-3-[1-((1R,5S)-6,6-dimethylbicyclo[3.1.1]hept-2-en-2-ylmethyl)piperidin-4-yl]urea;
′1-(3,5-Bistrifluoromethylphenyl)-3-[1-((1R,5S)-6,6-dimethylbicyclo[3.1.1]hept-2-en-2-ylmethyl)piperidin-4-yl]urea;
′1-[1-((1R,5S)-6,6-Dimethylbicyclo[3.1.1]hept-2-en-2-ylmethyl)piperidin-4-yl]-3-(3-trifluoromethylphenyl)urea;
′1-(4-Chloro-3-trifluoromethylphenyl)-3-[1-((E)-1-cyclooct-1-enyl)methyl-piperidine-4-yl]urea;
′1-(3,5-Bistrifluoromethylphenyl)-3-[1-((E)-1-cyclooct-1-enyl)methyl-piperidin-4-yl]urea
′1-[1-((E)-1-Cyclooct-1-enyl)methylpiperidin-4-yl]-3-(3-ethylphenyl)urea;
and the salts, solvates, hydrates, tautomers or N-oxides thereof.

Compounds according to the invention are potent and selective inhibitors of chemokines binding to the CXCR3 receptor as demonstrated by differential inhibition of this receptor when compared to other chemokine receptors, such as CCR3. The ability of the compounds to act in this way may be simply determined by employing tests such as those described in the Examples hereinafter.

The compounds are of use in modulating chemokine mediated cell signalling and in particular are of use in the prophylaxis and/or treatment of diseases or disorders involving inappropriate T-cell trafficking. The invention extends to such a use and to the use of the compounds of formula (1) for the manufacture of a medicament for treating such diseases and disorders. Particular diseases include inflammatory, autoimmune and immunoregulatory disorders.

Particular uses to which the compounds of the invention may be put include: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scieroderma; respiratory allergic diseases such as asthma, allergic rhinitis, hypersensitivity lung diseases and the like. (2) autoimmune diseases, such as arthritis (rheumatoid and psoriatic), multiple sclerosis, systemic lupus erythematosus, diabetes, glomerulonephritis and the like. (3) graft rejection (including allograft rejection and graft-v-host disease), and (4) other diseases in which undesired inflammatory responses are to be inhibited e.g. atherosclerosis, myositis, neurodegenerative diseases, Alzheimer's disease, encephalitis, meningitis, hepatitis, nephritis, sepsis, sarcoidosis, conjunctivitis, otitis, chronic obstructive pulmonary disease, sinusitis, Behcet's syndrome, Sjorgen's syndrome and glomerulonephrites.

In a particular embodiment, the compounds of the present invention are useful for the treatment of the aforementioned exemplary disorders irrespective of their etiology, for example, for the treatment of multiple sclerosis, psoriasis, rheumatoid arthritis, allograft rejection and graft-v-host disease.

The compounds of formula (1) can be used alone or in combination with other compounds having related utilities to prevent and treat inflammatory and immunoregulatory disorders and diseases, including asthma and allergic diseases, as well as autoimmune pathologies such as multiple sclerosis, rheumatoid arthritis and atherosclerosis, and those pathologies as discussed herein.

For the prophylaxis or treatment of disease the compounds according to the invention may be administered as pharmaceutical compositions, and according to a further aspect of the invention we provide a pharmaceutical composition which comprises a compound of formula (1) together with one or more pharmaceutically acceptable carriers, excipients or diluents.

Alternate compositions of this invention comprise a compound of formula (1) or a salt thereof; an additional agent selected from an immunosuppressant or an anti-inflammatory agent; and any pharmaceutically acceptable carrier, adjuvant or vehicle.

Pharmaceutical compositions according to the invention may take a form suitable for oral, buccal, parenteral, nasal, topical, vaginal or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); -lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound

For buccal administration the compositions may take the form of tablets or lozenges formulated in conventional manner.

The compounds for formula (1) may be formulated for parenteral administration by injection e.g. by bolus injection or infusion. Formulations for injection may be presented in unit dosage form, e.g. in glass ampoule or multi dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use. For particle mediated administration the compounds of formula (1) may be coated on particles such as microscopic gold particles.

In addition to the formulations described above, the compounds of formula (1) may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichloro-fluoromethane, dichlorotetrafluoroethane, carbon dioxide or other suitable gas or mixture of gases.

For vaginal or rectal administration the compounds of formula (1) may be formulated as a suppository. These formulations may be prepared by mixing the active ingredient with a suitable non-irritating excipient which is a solid at room temperature but liquid at the body temperature. Such materials include for example cocoa butter and polyethylene glycols.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg e.g. around 0.01 mg/kg to 40 mg/kg body weight for oral or buccal administration, from around 10 ng/kg to 50 mg/kg body weight for parenteral administration and around 0.05 mg to around 1000 mg e.g. around 0.5 mg to around 1000 mg for nasal administration or administration by inhalation or insufflation.

The compounds of the invention may be prepared by a number of processes as generally described below and more specifically in the Examples hereinafter. Many of the reactions described are well-known standard synthetic methods which may be applied to a variety of compounds and as such can be used not only to generate compounds of the invention, but also where necessary the intermediates thereto.

In the following process description, the symbols D, E, Alk³, n, m, R¹ and R² when used in the formulae depicted are to be understood to represent those groups described above in relation to formula (1) unless otherwise indicated. In the reactions described below, it may be necessary to protect reactive functional groups, for example hydroxy, amino, thio or carboxy groups, where these are desired in the final product, to avoid their unwanted participation in the reactions. Conventional protecting groups may be used in accordance with standard practice [see, for example, Green, T. W. in “Protective Groups in Organic Synthesis”, John Wiley and Sons, (1999) and the examples herein]. In some instances, deprotection may be the final step in the synthesis of a compound of formula (1) and the processes according to the invention described hereinafter are to be understood to extend to such removal of protecting groups.

Thus according to a further aspect of the invention, a compound of formula (1) may be prepared from an amine of general formula (i) using the general method as shown in Scheme A:

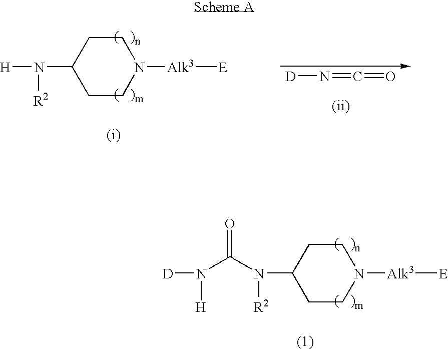

Thus, an amine of formula (i) may be reacted with an isocyanate of general formula (ii) in the presence of a base, such as an amine base e.g. triethylamine or diisopropylethylamine in a solvent such as a halogenated hydrocarbon e.g. dichloromethane at around ambient temperature to give a compound of general formula (1) where R¹ is a hydrogen atom.

The amine of general formula (i) may be prepared using the general Scheme B as shown below:

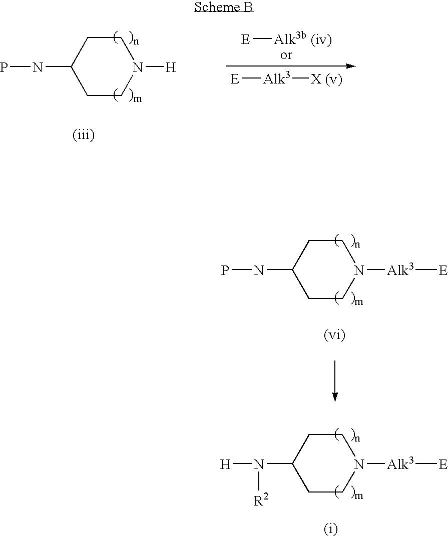

Thus, an amine of general formula (iii) where P is a suitable protecting group e.g. tert-butoxycarbonyl, may be reacted with a compound of formula E-Alk³-X (v), wherein X is a suitable leaving group (e.g. a halogen, such as chlorine or bromine, or an arylsulfonyloxy group, such as p-toluene sulfonate) to give a compound of general formula (vi). The reaction may be performed in the presence of a base, such as potassium carbonate in, for example, refluxing acetonitrile or N,N-dimethylformamide at around ambient temperature.

Alternatively the protected amine of general formula (vi) may be prepared by reductive alkylation of a compound of formula (iii) with a compound of formula E-Alk^{3b} (iv), wherein Alk^{3b} is a suitable precursor to Alk³, for example Alk^{3b} contains a reactive group, such as a reactive carbonyl. This reaction may be achieved using methods known to those skilled in the art. For example, when Alk^{3b} is an aldehyde, appropriate conditions may include the use of a suitable borohydride as reductant, for example sodium triacetoxyborohydride or sodium cyanoborohydride, in a solvent such as a halogenated hydrocarbon, e.g. dichloromethane, or an alcohol, e.g. methanol or ethanol, where necessary in the presence of an acid such as acetic acid at around ambient temperature. A dehydrating agent, such as an orthoformate e.g. triethylorthoformate or trimethylorthoformate may also be employed in the reaction.

The compounds of formula (v) may be prepared from an alcohol of general formula E-Alk³-OH (vii) using standard methodology known to those skilled in the art. For example, when X is an arylsulfonate ester, this may be prepared by reaction of the alcohol (vii) with p-toluenesulfonyl chloride in the presence of an amine base, e.g. triethylamine in an appropriate solvent, such as dichloromethane or tetrahydrofuran.

The compounds of formula (vii) may also be used to prepare the compounds of formula (iv) using standard oxidising conditions such as those described herein.

The intermediate compound of formula (vi) may be deprotected using standard methodology, for example by treatment with an acid such as trifluoroacetic acid or hydrochloric acid, to give an amine starting material of general formula (i) wherein R² is a hydrogen atom. This may be alkylated using standard techniques known to those skilled in the art, such as those methods as described herein, to give an amine of formula (vi) wherein R² is an alkyl group.

A compound of formula (1) may also be prepared by the general method as shown in Scheme C:

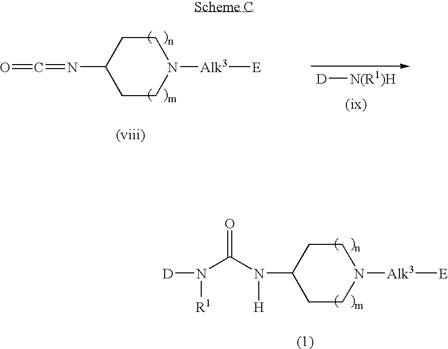

Thus an isocyanate of formula (viii) may be reacted with an amine of formula (ix) in the presence of a base, such as an amine base e.g. triethylamine or diisopropylethylamine in a solvent such as a halogenated hydrocarbon e.g. dichloromethane to give a compound of general formula (1) where R² is a hydrogen atom.

It will be further appreciated that the order of reactions in which a compound of formula (1) is prepared may be varied. Thus, for example, an amine of formula (x): 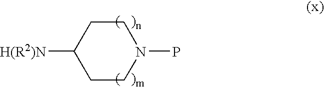
where P is as defined above, may be reacted with an isocyanate of general formula (ii) using the reactions just described to yield a compound of formula (xi). Alternatively an amine of formula (x), wherein R² is a hydrogen atom, may be converted into an isocyanate, for example, using an appropriate reagent such as triphosgene or trichloromethyl chloroformate using conditions known to those skilled in the art, and subsequently reacted with an amine of formula (ix). The resulting urea of formula (xi): 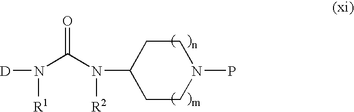
may be deprotected using methods known to those skilled and reacted with a compound of general formula (iv) or (v) using standard techniques, such as the methods described herein.

The synthesis of compounds of formula (1) may be amenable to high throughput methods, such as combinatorial or parallel synthesis techniques familiar to those skilled in the art.

Intermediates of formulae (i)-(xi) and any other intermediates required to obtain compounds of formula (1), if not available commercially, may be prepared by methods known to those skilled in the art following procedures set forth in references such as Rodd's Chemistry of Carbon Compounds, Volumes 1-15 and Supplementals (Elsevier Science Publishers, 1989), Fieser and Fieser's Reagents for Organic Synthesis, Volumes 1-19 (John Wiley and Sons, 1999), Comprehensive Heterocyclic Chemistry, Ed. Katritzky et al, Volumes 1-8, 1984 and Volumes 1-11, 1994 (Pergamon), Comprehensive Organic Functional Group Transformations, Ed. Katritzky et al, Volumes 1-7, 1995 Pergamon), Comprehensive Organic Synthesis, Ed. Trost and Flemming, Volumes 1-9, (Pergamon, 1991), Encyclopedia of Reagents for Organic Synthesis Ed. Paquette, Volumes 1-8 (John Wiley and Sons, 1995), Larock's Comprehensive Organic Transformations (VCH Publishers Inc., 1989) and March's Advanced Organic Chemistry (John Wiley and Sons, 1992).

For example, an isocyanate of general formula (ii) or (viii) may be prepared by reacting an appropriate amine precursor with an appropriate reagent such as triphosgene or trichloromethyl chloroformate using conditions known to those skilled in the art.

The amine precursors of formulae (i), (iii), (ix) or (x) when not commercially available may be prepared using well-known literature methods.

It will be appreciated that compounds of formula (1), or any preceding intermediates may be further derivatised by one or more standard synthetic methods employing substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other compounds of formula (1), where appropriate functional groups exist in these compounds.

For example, ester groups may be converted to the corresponding acid [—CO₂H] by acid- or base-catalysed hydrolysis depending on the nature of the ester Acid- or base-catalysed hydrolysis may be achieved for example by treatment with an organic or inorganic acid, e.g. trifluoroacetic acid in an aqueous solvent or a mineral acid such as hydrochloric acid in a solvent such as dioxan or an alkali metal hydroxide, e.g. lithium hydroxide in an aqueous alcohol, e.g. aqueous methanol. Similarly an acid [—CO₂H] may be prepared by hydrolysis of the corresponding nitrile [—CN], using for example a base such as sodium hydroxide in a refluxing alcoholic solvent, such as ethanol.

In another example, —OH groups may be generated from a corresponding ester or aldehyde [—CHO] by reduction, using for example a complex metal hydride such as lithium aluminium hydride or sodium borohydride in a solvent such as methanol. Alternatively an alcohol may be prepared by reduction of the corresponding acid [—CO₂H], using for example lithium aluminium hydride in a solvent such as tetrahydrofuran.

Alcohol groups may be converted into leaving groups, such as an halogen atoms or sulfonyloxy groups such as an alkylsulfonyloxy, e.g. trifluoromethylsulfonyloxy or arylsulfonyloxy, e.g. p-toluenesulfonyloxy group using conditions known to the skilled artisan. For example, an alcohol may be reacted with thionyl chloride in a halogenated hydrocarbon e.g., dichloromethane to yield the corresponding chloride. A base e.g., triethylamine may also be used in the reaction.

Aldehyde [—CHO] groups may be obtained by oxidation of a corresponding alcohol using well known conditions. For example using an oxidising agent such as a periodinane e.g Dess Martin, in a solvent such as a halogenated hydrocarbon, e.g. dichloromethane. An alternative oxidation may be suitably activating dimethyl sulfoxide using for example, oxalyl chloride, followed by addition of an alcohol, and subsequent quenching of the reaction by the addition of an amine base, such as triethylamine. Suitable conditions for this reaction may be using an appropriate solvent, for example, a halogenated hydrocarbon, e.g. dichloromethane at −78° C. followed by subsequent warming to room temperature.

α,β-Unsaturated aldehydes, for example, of formula OHCE, where E is cycloalkenyl, may be prepared by hydrolysis of a corresponding allylic nitro compound. This may be achieved, for example, by treatment of the allylic nitro compound with a base, such as sodium methoxide or potassium tert-butoxide, followed by addition of a buffered aqueous titanium trichloride solution. The allylic nitro compound may be prepared by nucleophilic addition of nitromethane to the corresponding ketone, followed by elimination of water. Suitable conditions for this reaction may be refluxing in toluene under Dean Stark conditions, in the presence of an amine base, such as N,N-dimethylethylene diamine. It will be appreciated that these aldehydes may be used in reductive alkylations to give compounds of formula (1) where Alk³ is —CH₂— using the conditions described herein.

In a further example primary amine (—NH₂) or secondary amine (—NH—) groups may be alkylated using a reductive alkylation process employing an aldehyde and a borohydride, for example sodium triacetoxyborohyride or sodium cyanoborohydride, in a solvent such as a halogenated hydrocarbon, e.g. dichloromethane, a ketone such as acetone, or an alcohol, e.g. ethanol, where necessary in the presence of an acid such as acetic acid at around ambient temperature.

In a further example, amine [—NH₂] groups may be obtained by hydrolysis from a corresponding imide by reaction with hydrazine in a solvent such as an alcohol, e.g. ethanol at ambient temperature.

In another example, a nitro [—NO₂] group may be reduced to an amine [—NH₂], for example by catalytic hydrogenation using for example hydrogen in the presence of a metal catalyst, for example palladium on a support such as carbon in a solvent such as an ether, e.g. tetrahydrofuran or an alcohol e.g. methanol, or by chemical reduction using for example a metal, e.g. tin or iron, in the presence of an acid such as hydrochloric acid.

In a further example amine (—CH₂NH₂) groups may be obtained by reduction of nitriles (—CN), for example by catalytic hydrogenation using for example hydrogen in the presence of a metal catalyst, for example palladium on a support such as carbon, or Raney® nickel, in a solvent such as an ether e.g. a cyclic ether such as tetrahydrofuran or an alcohol, e.g. methanol or ethanol, optionally in the presence of ammonia solution at a temperature from ambient to the reflux temperature, or by chemical reduction using for example a metal hydride, e.g. lithium aluminium hydride, in a solvent such as an ether, e.g. a cyclic ether such as tetrahydrofuran, at a temperature from 0° C. to the reflux temperature.

Aromatic halogen substituents in the compounds may be subjected to halogen-metal exchange with a base, for example a lithium base such as n-butyl or t-butyl lithium, optionally at a low temperature, e.g. around −78° C., in a solvent such as tetrahydrofuran and then quenched with an electrophile to introduce a desired substituent. Thus, for example, a formyl group may be introduced by using dimethylformamide as the electrophile; a thiomethyl group may be introduced by using dimethyldisulphide as the electrophile.

N-oxides of compounds of formula (1) may be prepared for example by oxidation of the corresponding nitrogen base using an oxidising agent such as hydrogen peroxide in the presence of an acid such as acetic acid, at an elevated temperature, for example around 70° C. to 80° C., or alternatively by reaction with a peracid such as peracetic acid in a solvent, e.g. dichloromethane, at ambient temperature.

Salts of compounds of formula (1) may be prepared by reaction of a compound of formula (1) with an appropriate base or acid in a suitable solvent or mixture of solvents e.g. an organic solvent such as an ether e.g. diethylether, or an alcohol, e.g. ethanol or an aqueous solvent using conventional procedures. Salts of compounds of formula (1) may be exchanged for other salts by use of conventional ion-exchange chromatography procedures.

Where it is desired to obtain a particular enantiomer of a compound of formula (1) this may be produced from a corresponding mixture of enantiomers using any suitable conventional procedure for resolving enantiomers.

Thus for example diastereomeric derivatives, e.g. salts, may be produced by reaction of a mixture of enantiomers of formula (1) e.g. a racemate, and an appropriate chiral compound, e.g. a chiral base. The diastereomers may then be separated by any convenient means, for example by crystallisation and the desired enantiomer recovered, e.g. by treatment with an acid in the instance where the diastereomer is a salt.

In another resolution process a racemate of formula (1) may be separated using chiral High Performance Liquid Chromatography. Alternatively, if desired a particular enantiomer may be obtained by using an appropriate chiral intermediate in one of the processes described above.

Chromatography, recrystallisation and other conventional separation procedures may also be used with intermediates or final products where it is desired to obtain a particular geometric isomer of the invention.

The following Examples illustrate the invention. All temperatures are in ° C. Where experimental detail is not given for the preparation of a reagent it is either commercially available, or it is known in the literature, for which the CAS number is quoted. The compounds are named with the aid of Beilstein Autonom supplied -by MDL Information Systems GmbH, Theodor-Heuss-Allee 108, D-60486-Frankfurt, Germany. ¹H NMR spectra were obtained at 300 MHz or 400 MHz unless otherwise indicated.

The following LCMS conditions were used to acquire the retention times as reported herein:

### LCMS Conditions:

HP1100 (Diode Array) linked to a Finnigan LcQ Duo Mass Spectrometer.

| Column: | Luna C18(2) 100 × 4.6 mm, 5 μn particle size | |
|---|---|---|
| | Analytical column | |
| Column Temp: | 35° C. | |
| Mobile Phase: | A: 0.08% formic acid in H₂O | |
| | B: 0.08% formic acid in MeCN | |
| Flow rate: | 3 ml/min | |
| Gradient: | Time (mins): | % Composition B: |
| | 0.0 | 95.0 |
| | 4.40 | 5.0 |
| | 5.30 | 5.0 |
| | 5.32 | 95.0 |
| | 6.50 | 95.0 |

| | |
|---|---|
| Run time: | 6.50 mins |
| Typical Injection Vol: | 10 μl |
| Detector Wavelength: | 210 nm |

### Preparative LC Conditions (HPLC):

MassLynx Setup

| | |
|---|---|
| Column: | Luna C18(2) 100 × 21.2 mm, 5 μn |
| | particle size PREP |
| Column Temp: | Ambient |
| Mobile Phase: | A: Water + 0.08% formic acid |
| | B: Acetonitrile + 0.08% formic acid |
| Gradient: | Variable - depends on retention of |
| | sample in LCMS screen |
| Run Time: | 10 mins |
| Flow rate: | 20 ml/min |
| Typical Injection Vol: | 0.8 ml of 20 mg/ml solution |
| Detector Wavelength: | 210 and 254 nm |

Abbreviations used:

| | |
|---|---|
| DCM - Dichloromethane | THF - Tetrahydrofuran |
| MeOH - Methanol | EtOAc - Ethyl acetate |
| TFA - Trifluoroacetic acid | BOC - tert-butoxycarbonyl |
| CDCl₃ - Deuterated chloroform | DMSO-d₆ - Deuterated |
| | dimethylsulfoxide |
| Methanol-d₄ - Deuterated methanol | DMF - N,N-dimethylformamide |

### Intermediate 1

### 4-(Boc-amino)-1-cycloocten-1-yl piperidine

Piperidin-4-yl-carbamic acid tert-butyl ester hydrochloride [CAS No. 73874-95-0] (2 g) was dissolved in DCM (20 ml) and triethylamine (2 g) and triethylorthoformate (5 ml) were added. 1-Cyclooctene carboxaldehyde [CAS No. 6038-12-6] (2 g) was added and the mixture stirred for 30 min, then sodium triacetoxyborohydride (4 g) was added and the mixture stirred overnight at room temperature. The solution was washed with sodium bicarbonate (20 ml), dried (MgSO₄) and evaporated to give the title compound as a beige solid (2.6 g). TLC R_{f} 0.25 (5% MeOH/DCM)

Intermediate 2 was prepared in a similar manner to Intermediate 1:

### Intermediate 2

### [1-(6,6-Dimethylbicyclo[3.1.1]hept-2-en-2-ylmethyl)piperidin-4-yl]-carbamic acid tert-butyl ester

From piperidin-4-yl-carbamic acid tert-butyl ester hydrochloride [CAS No. 73874-95-0] (1.63 g) and 6,6-dimethylbicyclo[3.1.1]hept-2-ene-2-carbaldehyde [CAS No. 18486-69-6] (1.26 ml) to give a light yellow oil (2.91 g). Purification by column chromatography (5% MeOH/DCM) afforded the title compound as colourless solid (1.75 g). Retention time 2.25 minutes. M+H 335

### Intermediate 3

### 4-Amino-1-cycloocten-1-ylpiperidine

TFA (10 ml) was added to a solution of Intermediate 1 (2.6 g) in DCM (30 ml) at room temperature. The solution was stirred for 2 h, and then evaporated in vacuo and the residue dissolved in water (30 ml) and washed with ether (20 ml). The aqueous layer was basified with sodium hydroxide pellets and extracted with DCM (2×20 ml). The solvent was washed with water (20 ml) and brine (20 ml), dried (MgSO₄) and evaporated to give the title compound as a pale yellow oil. TLC R_{f} 0.22 (10% MeOH/DCM 1% NH₄OH).

Intermediate 4 was prepared in a similar manner to Intermediate 3:

### Intermediate 4

### 1-(6,6-Dimethylbicyclo[3.1.1]hept-2-en-2-ylmethyl)piperidin-4-ylamine

From Intermediate 2 (3 g.) to give the title compound as an orange oil (2.4 g). TLC R_{f} 0.30 (10% MeOH/DCM 1% NH₄OH).

### Intermediate 5

### [1-(6,6-Dimethylbicyclo[3.1.1]hept-2-en-2-ylmethyl)piperidin-4-yl]methyl-amine

Intermediate 2 (368 mg) was dissolved in THF (5.0 ml) and cooled to 0° C. LiAlH₄ (1.0 M, 14 ml) solution in THF was added and the reaction was stirred overnight at room temperature. Isopropanol (about 5 ml) was carefully added followed by H₂O (0.156 ml), 15% NaOH (0.156 ml) and H₂O (0.469 ml). After stirring for 1 hr. the grey precipitate was filtered off and the filtrate concentrated to give the title compound as a yellow oil (250 mg). Retention time 1.10 minutes. M+H 249

### Intermediate 6

### N-[1-(6,6-Dimethylbicyclo[3.1.1]hept-2-en-2-ylmethyl)piperidin-4-yl]-acetamide

Intermediate 4 (992 mg) was dissolved in DCM (25 ml) and diisopropylethylamine (0.697 ml) was added. The reaction mixture was cooled to 0° C. and acetyl chloride (0.213 ml) was added drop wise. Stirring was continued overnight at room temperature. The reaction mixture was extracted with sodium bicarbonate (2×20 ml), brine (20 ml), dried (MgSO₄) and evaporated to give the title compound as a white solid (0.85 g). Retention time 1.64 minutes. M+H 277

### Intermediate 7

### [1-(6,6-Dimethylbicyclo[3.1.1]hept-2-en-2-ylmethyl)piperidin-4-yl]ethyl-amine

Intermediate 6 (850 mg) was dissolved in THF (20.0 ml) and cooled to 0° C. LiAIH₄ (1.0 M, 10 ml) solution in THF was added and the reaction was stirred overnight at room temperature. Isopropanol (about 5 ml) was carefully added followed by H₂O (0.380 ml), 15% NaOH (0.380 ml) and H₂O (1.14 ml). After stirring for 1 hr. the grey precipitate was filtered off and the filtrate concentrated to give the title compound as a yellow oil (590 mg). Retention time 1.05 minutes. M+H 263

### Intermediate 8

### 1-Naphthalen-2-yl-3-piperidin-4-yl-urea hydrochloride

2-Naphthyl isocyanate (432 mg) was added to a solution of boc-(4-amino)-piperidine hydrochloride in anhydrous DCM. Triethylamine (360 μl) was added and the reaction mixture was stirred for 17 h at room temperature. The reaction mixture was washed with 0.5N HCl, then saturated aqueous NaHCO₃ solution, dried (MgSO₄) and concentrated in vacuo to yield 4-(3-naphthalen-2-yl-ureido)-piperidine-1-carboxylic acid tert-butyl ester as an off-white powder.

To a solution of this product in methanol (14 ml) was added a 1M solution of HCl in diethyl ether (10 ml) and the reaction mixture was stirred for 18 h at room temperature. The solvent was removed in vacuo and the residue was triturated with diethyl ether and dried in vacuo to afford the title compound as a beige powder (674 mg). LCMS m/z 270 (MH⁺) observed.

### EXAMPLE 1

### N-2-Naphthyl-N′-(cyclooctene-1-yl)methylpiperidine urea

2-Naphthyl isocyanate (1 g) was added to a solution of tert-butyl 4-aminopiperidine-1-carboxylate (CAS No. 73874-95-0) (1.2 g) in DCM and the solution was stirred for 24 h at room temperature. The mixture was evaporated in vacuo and the solid product triturated with ether. The residue was dissolved in DCM (50 ml) and TFA (10 ml) was added. The solution was stirred for 3 h, then evaporated in vacuo and the residue crystallised from methanol (5 ml)/diethyl ether (20 ml). The solid product was dissolved in DCM (50 ml) and trimethyl orthoformate (10 ml) and triethylamine (1.5 ml) were added, followed by 1-cyclooctene carboxaldehyde (1.2 g). The mixture was stirred for 1 h, then sodium triacetoxyborohydride (3 g) was added. The resulting suspension was stirred overnight, then the mixture was filtered through Celite®, washed with water (20 ml) and sodium bicarbonate (20 ml) solution and evaporated. The residue was crystallised from EtOAc/hexanes to give the title compound as colourless solid (0.85 g). TLC R_{f} 0.35 (10% MeOH/DCM). MS 391 (M⁺)

### EXAMPLE 2

### 1-[1-((E)-1-Cyclooct-1-enyl)methylpiperidin-4-yl]-3-(3,4-dichlorophenyl)-urea

3,4-Dichlorophenyl isocyanate (100 mg) was added to a solution of Intermediate 3 (100 mg) in DCM (10 ml). Triethylamine (100 mg) was added and the solution was stirred overnight, washed with water (10 ml) and brine (10 ml), then evaporated to dryness and triturated with ether to give the title compound as colourless solid (0.15 g). Retention time 2.39 minutes. TLC R_{f} 0.40 (10% MeOH/DCM). MS 410 (M+1)

### EXAMPLE 3

### 1-[1-((E)-1-Cyclooct-1-enyl)methylpiperidin-4-yl]-3-(4-trifluoromethylphenyl)urea

4-Trifluoromethylphenyl isocyanate (100 mg) was added to a solution of Intermediate 3 (100 mg) in DCM (10 ml). Triethylamine was added and the mixture stirred overnight, washed with water (10 ml) and brine (10 ml), dried (MgSO₄) and evaporated and the residue triturated with ether to give the title compound as colourless solid (0.12 g). Retention time 2.36 minutes. TLC R_{f} 0.29 (10% MeOH/DCM). MS 410 (M+1)

The compounds of :Examples 4-11 were prepared in a similar manner to the compound of Example 3 using Intermediate 3 and the appropriate commercially available isocyanate:

### EXAMPLE 4

### 1-(3-Cyanophenyl)-3-[1-((E)-1-cyclooct-1-enyl)methylpiperidin-4-yl]urea

From 3-cyanophenyl isocyanate. Yield 7.5 mg.

Retention time 2.06 minutes. TLC R_{f} 0.30 (10% MeOH/DCM). MS 367 M+1

### EXAMPLE 5

### 1-Benzo[1,3]dioxol-5-yl-3-[1-((E)-1-cyclooct-1-enyl)methylpiperidin-4-yl]-urea

From 3,4-methylenedioxyphenyl isocyanate. Yield 15 mg.

Retention time 2.01 minutes. TLC R_{f} 0.26 (10% MeOH/DCM). MS 386 M+1

### EXAMPLE 6

### 1-[1-((E)-1-Cyclooct-1-enyl)methylpiperidin-4-yl]-3-(4-phenoxyphenyl)-urea

From 4-phenoxyphenyl isocyanate. Yield 9 mg.

Retention time 2.43 minutes. TLC R_{f} 0.37 (10% MeOH/DCM). MS 434 (M+1)

### EXAMPLE 7

### 1-Biphenyl-4-yl-3-[1-((E)-1-cyclooct-1-enyl)methylpiperidin-4-yl]-urea

From biphenyl isocyanate. Yield 8.5 mg.

Retention time 2.45 minutes. TLC R_{f} 0.37 (10% MeOH/DCM). MS 418 (M+1)

### EXAMPLE 8

### 1-[1-((E)-1-Cyclooct-1-enyl)methylpiperidin-4-yl]-3-(2,2,4,4-tetrafluoro-4H-benzo[1,3]dioxin-6-yl)urea

From 6-isocyano-2,2,4,4-tetrafluoro-1,3-benzodioxane. Yield 22 mg.

Retention time 2.53 minutes. TLC R_{f} 0.40 (10% MeOH/DCM). MS 472 (M+1)

### EXAMPLE 9

### 1-[1-((E)-1-Cyclooct-1-enyl)methylpiperidin-4-yl-3-indan-5-ylurea

From 5-indanyl isocyanate. Yield 11 mg.

Retention time 2.27 minutes. TLC R_{f} 0.33 (10% MeOH/DCM). MS 382 (M+1)

### EXAMPLE 10

1-(4-Cyanophenyl)-3-[1-((E)-1-cyclooct-1-enyl)methylpiperidin-4-yl]urea

From 4-cyanophenyl isocyanate. Yield 12 mg.

Retention time 2.06 minutes. TLC R_{f} 0.25 (10% MeOH/DCM). MS 367 (M+1)

### EXAMPLE 11

### 1-[-(6,6-Dimethylbicyclo[3.1.1]hept-2-en-2-ylmethyl)piperidin-4-yl]-3-naphthalen-2-yl urea

Prepared from Intermediate 4 (100 mg) and 2-naphthyl isocyanate (100 mg) to afford the title compound as a white solid 0.13 g. TLC R_{f} 0.37 (10% MeOH/DCM). MS 404 (M+1)

### EXAMPLE 12

### 1-[1-(6,6-Dimethylbicyclo[3.1.1]hept-2-en-2-ylmethyl)piperidin-4-yl]-1-methyl-3-(3-trifluoromethylphenyl)urea

To trifluorometatolyl isocyanate (38 mg) was added 1.0 ml of a 0.2 M solution of Intermediate 5 in dry DCM. The title compound was obtained by concentration of the reaction mixture and purification by prep HPLC. (32 mg). Retention time 2.46 minutes. M+H 436

Examples 13-47 were prepared in a similar manner to the compound of Example 12 from commercially available isocyanates using solution phase parallel synthesis methodology:

### EXAMPLE 13

### 1-[1-(6,6-Dimethylbicyclo[3.1.1]hept-2-en-2-ylmethyl)piperidin-4-yl]-1-methyl-3-naphthalen-2-yl urea

From 2-naphthyl isocyanate (34 mg) and 1.0 ml of a 0.2 M solution of Intermediate 5 in dry DCM. Yield 21 mg. Retention time 2.39 minutes. M+H 418

### EXAMPLE 14

### 1-[1-(6,6-Dimethylbicyclo[3.1.1]hept-2-en-2-ylmethyl)piperidin-4-yl]-1-methyl-3-(3-methylsulfanylphenyl)urea

From 3-(methylthio)phenyl isocyanate (33 mg) and 1.0 ml of a 0.2 M solution of Intermediate 5 in dry DCM. Yield 45 mg. Retention time 2.29 minutes. M+H 414

### EXAMPLE 15

### 1-[1-(6,6-Dimethylbicyclo[3.1.1]hept-2-en-2-ylmethyl)piperidin-4-yl]-3-(3-ethylphenyl)-1-methylurea

From 3-ethylphenyl isocyanate (29 mg) and 1.0 ml of a 0.2 M solution of Intermediate 5 in dry DCM. Yield 40 mg. Retention time 2.36 minutes. M+H 396

### EXAMPLE 16

### 1-[1-(6,6-Dimethylbicyclo[3.1.1]hept-2-en-2-ylmethyl)piperidin-4-yl]-1-methyl-3-(5-phenylthiophen-2-yl)urea

From 5-phenyl-2-thienyl isocyanate (40 mg) and 1.0 ml of a 0.2 M solution of Intermediate 5 in dry DCM. Yield 42 mg. Retention time 2.54 minutes. M+H 450

### EXAMPLE 17

### 1-(1-Adamantan-1-ylmethylpiperidin-4-yl)-3-(3-trifluoromethylphenyl)-urea

From trifluorometatolyl isocyanate (76 mg) and 2.0 ml of a 0.2 M solution of 1-adamantan-1-ylmethylpiperidin-4-ylamine [CAS No. 64306-80-5] in dry DCM. Yield 45 mg. Retention time 2.38 minutes. M+H 436

### EXAMPLE 18

### 1-(1-Adamantan-1-ylmethylpiperidin-4-yl)-3-naphthalen-2-ylurea

From 2-naphthyl isocyanate (68 mg) and 2.0 ml of a 0.2 M solution of 1-adamantan-1-ylmethylpiperidin-4-ylamine [CAS No. 64306-80-5] in dry DCM. Yield 29 mg. Retention time 2.38 minutes. M+H 418

### EXAMPLE 19

### 1-(1-Adamantan-1-ylmethylpiperidin-4-yl)-3-(3-methylsulfanylphenyl)-urea

From 3-(methylthio)phenyl isocyanate (66 mg) and 2.0 ml of a 0.2 M solution of 1-adamantan-1-ylmethyl-piperidin-4-ylamine [CAS No. 64306-80-5] in dry DCM. Yield 19 mg. Retention time 2.31 minutes. M+H 414

### EXAMPLE 20

### 1-(1-Adamantan-1-ylmethylpiperidin-4-yl)-3-(3-ethylphenyl)urea

From 3-ethylphenyl isocyanate (58 mg) and 2.0 ml of a 0.2 M solution of 1-adamantan-1-ylmethylpiperidin-4-ylamine [CAS No. 64306-80-5] in dry DCM. Yield 9 mg. Retention time 2.38 minutes. M+H 396

### EXAMPLE 21

### 1-(1-Adamantan-1-ylmethylpiperidin-4-yl)-3-(5-phenylthiophen-2-yl)urea

From 5-phenyl-2-thienyl isocyanate (80 mg) and 2.0 ml of a 0.2 M solution of 1-adamantan-1-ylmethylpiperidin-4-ylamine [CAS No. 64306-80-5] in dry DCM. Yield 20 mg. Retention time 2.56 minutes. M+H 450

### EXAMPLE 22

### 1-[1-(6,6-Dimethylbicyclo[3.1.1]hept-2-en-2-ylmethyl)piperidin-4-yl]-1-ethyl-3-(3-trifluoromethylphenyl)urea

From trifluorometatolyl isocyanate (14 mg) and 1.0 ml of a 0.075 M solution of Intermediate 7 in dry DCM. Yield 7 mg. Retention time 2.58 minutes. M+H 450

### EXAMPLE 23

### 1-[1-(6,6-Dimethylbicyclo[3.1.1]hept-2-en-2-ylmethyl)piperidin-4-yl]-1-ethyl-3-naphthalen-2-ylurea

From 2-naphthyl isocyanate (13 mg) and 1.0 ml of a 0.075 M solution of Intermediate 7 in dry DCM. Yield 13 mg. Retention time 2.54 minutes. M+H 432

### EXAMPLE 24

### 1-[1-(6,6-Dimethylbicyclo[3.1.1]hept-2-en-2-ylmethyl)piperidin-4-yl]-1-ethyl-3-(3-methylsulfanylphenyl)urea

From 3-(methylthio)phenyl isocyanate (12 mg) and 1.0 ml of a 0.075 M solution of Intermediate 7 in dry DCM. Yield 13 mg. Retention time 2.40 minutes. M+H 428

### EXAMPLE 25

### 1-[1-(6,6-Dimethylbicyclo[3.1.1]hept-2-en-2-ylmethyl)piperidin-4-yl]-1-ethyl-3-(3-ethylphenyl)urea

From 3-ethylphenyl isocyanate (11 mg) and 1.0 ml of a 0.075 M solution of Intermediate 7 in dry DCM. Yield 12 mg. Retention time 2.49 minutes. M+H 410

### EXAMPLE 26

### 1-[1-(6,6-Dimethylbicyclo[3.1.1]hept-2-en-2-ylmethyl)piperidin-4-yl]-1-ethyl-3-(5-phenylthiophen-2-yl)urea

From 5-phenyl-2-thienyl isocyanate (15 mg) and 1.0 ml of a 0.075 M solution of Intermediate 7 in dry DCM. Yield 10 mg. Retention time 2.70 minutes. M+H 464

### EXAMPLE 27

### 1-[1-(6,6-Dimethylbicyclo[3.1.1]hept-2-en-2-ylmethyl)piperidin-4-yl]-3-(3-ethylphenyl)urea

From 3-ethylphenyl isocyanate (300 mg) and Intermediate 4 (614 mg). Yield 600 mg. Retention time 2.38 minutes. M+H 382

### EXAMPLE 28

### ′1-[1-((1R,5S)-6,6-Dimethylbicyclo[3.1.1]hept-2-en-2-ylmethyl)piperidin-4-yl]-3-(3-methylsulfanylphenyl)urea

From 3-(methylthio)phenyl isocyanate (12 mg) and 1.0 ml of a 0.075 M solution of Intermediate 4. Yield 3.42 mg. Retention time 2.24 minutes. M+H 400

### EXAMPLE 29

### ′3-{3-[1-((1R,5S)-6,6-Dimethylbicyclo[3.1.1]hept-2-en-2-ylmethyl)-piperidin-4-yl]ureido}benzoic acid methyl ester

From 3-(methoxycarbonyl)phenyl isocyanate (13 mg) and 1.0 ml of a 0.075 M solution of Intermediate 4. Yield 3.05 mg. Retention time 2.15 minutes. M+H 412

### EXAMPLE 30

### ′1-[1-((1R,5S)-6,6-Dimethylbicyclo[3.1.1

hept-2-en-2-ylmethyl)piperidin-4-yl]-3-(4-isopropylphenyl)urea

From 4-isopropylphenyl isocyanate (12 mg) and 1.0 ml of a 0.075 M solution of Intermediate 4. Yield 4.74 mg. Retention time 2.39 minutes. M+H 396

### EXAMPLE 31

### ′1-(4-tert-Butylphenyl)-3-[1-((1R,5S)-6,6-dimethylbicyclo[3.1.1]hept-2-en-2-ylmethyl)piperidin-4-yl]urea

From 4-tert-butylphenyl isocyanate (13 mg) and 1.0 ml of a 0.075 M solution of Intermediate 4. Yield 4.86 mg. Retention time 2.51 minutes. M+H 410

### EXAMPLE 32

### ′1-[1-((1R,5S)-6,6-Dimethylbicyclo[3.1.1]hept-2-en-2-ylmethyl)piperidin-4-yl]-3-(5-phenylthiophen-2-yl)urea

From 5-phenyl-2-thienyl isocyanate (15 mg) and 1.0 ml of a 0.075 M solution of Intermediate 4. Yield 4.11 mg. Retention time 2.49 minutes. M+H 436

### EXAMPLE 33

### ′1-[1-((E)-1-Cyclooct-1-enyl)methylpiperidin4-yl]-3-(2,6-dichloropyridin-4-yl)urea

From 2,6-dichloro-4-pyridyl isocyanate (14 mg) and 1.0 ml of a 0.075 M solution of Intermediate 3. Yield 4.10 mg. Retention time 2.20 minutes. M+H 410

### EXAMPLE 34

### ′1-[1-((E)-1-Cyclooct-1-enyl)methylpiperidin-4-yl]-3-(5-phenylthiophen-2-yl)urea

From 5-phenyl-2-thienyl isocyanate (15 mg) and 1.0 ml of a 0.075 M solution of Intermediate 3. Yield 7.26 mg. Retention time 2.44 minutes. M+H 424

### EXAMPLE 35

### ′1-(3-Bromophenyl)-3-[1-((1R,5S)-6,6-dimethyl-bicyclo[3.1.1]hept-2-en-2-ylmethyl)piperidin-4-yl]urea

From 3-bromophenyl isocyanate (15 mg) and 1.0 ml of a 0.075 M solution of Intermediate 4. Yield 3.75 mg. Retention time 2.30 minutes. M+H 432

### EXAMPLE 36

### ′1-(2,3-Dichlorophenyl)-3-[1-((1R,5S)-6,6-dimethylbicyclo[3.1.1]hept-2-en-2-ylmethyl)piperidin-4-y]urea

From 2,3 dichlorophenyl isocyanate (14 mg) and 1.0 ml of a 0.075 M solution of Intermediate 4. Yield 5.45 mg. Retention time 2.37 minutes. M+H 422

### EXAMPLE 37

### ′1-(3-Chlorophenyl)-3-[1-((1R,5S)-6,6-dimethylbicyclo[3.1.1]hept-2-en-2-ylmethyl)piperidin-4-yl]urea

From 3-chlorophenyl isocyanate (12 mg) and 1.0 ml of a 0.075 M solution of Intermediate 4. Yield 4.77 mg. Retention time 2.30 minutes. M+H 388

### EXAMPLE 38

### ′1-(4-Chlorophenyl)-3-[1-((1R,5S)-6,6-dimethylbicyclo[3.1.1]hept-2-en-2-ylmethyl)piperidin-4-yl]urea

From 4-chlorophenyl isocyanate (12 mg) and 1.0 ml of a 0.075 M solution of Intermediate 4. Yield 3.92 mg. Retention time 2.28 minutes. M+H 388

### EXAMPLE 39

### ′1-(4-Chloro-3-trifluoromethylphenyl)-3-[1-((1R,5S)-6,6-dimethyl-bicyclo[3.1.1]hept-2-en-2-ylmethyl)piperidin-4-yl]urea

From 4-chloro-3-trifluoromethylphenyl isocyanate (17 mg) and 1.0 ml of a 0.075 M solution of Intermediate 4. Yield 4.29 mg. Retention time 2.55 minutes. M+H 456

### EXAMPLE 40

### ′1-(3,5-Bistrifluoromethylphenyl)-3-[1-((1R,5S)-6,6-dimethyl-bicyclo[3.1.1]hept-2-en-2-ylmethyl)piperidin-4-yl]urea

From 3,5-bis(trifluoromethyl)phenyl isocyanate (19 mg) and 1.0 ml of a 0.075 M solution of Intermediate 4. Yield 3.85 mg. Retention time 2.70 minutes. M+H 490

### EXAMPLE 41

### ′1-[1-((1R,5S)-6,6-Dimethylbicyclo[3.1.1]hept-2-en-2-ylmethyl)piperidin-4-yl]-3-(3-trifluoromethylphenyl)urea

From 3-trifluoromethylphenyl isocyanate (14 mg) and 1.0 ml of a 0.075 M solution of Intermediate 4. Yield 4.59 mg. Retention time 2.38 minutes. M+H 422

### EXAMPLE 42

### ′1-[1-((1R,5S)-6,6-Dimethylbicyclo[3.1.1]hept-2-en-2-ylmethyl)piperidin-4-yl]-3-(3-fluorophenyl)urea

From 3-fluorophenyl isocyanate (10 mg) and 1.0 ml of a 0.075 M solution of Intermediate 4. Yield 3.66 mg. Retention time 2.18 minutes. M+H 372

### EXAMPLE 43

### ′1-[1-((1R5S)-6,6-Dimethylbicyclo[3.1.1]hept-2-en-2-ylmethyl)piperidin-4-yl]-3-(3-methoxyphenyl)urea

From 3-methoxyphenyl isocyanate (11 mg) and 1.0 ml of a 0.075 M solution of Intermediate 4. Yield 6.60 mg. Retention time 2.12 minutes. M+H 384

### EXAMPLE 44

### ′1-(4-Chloro-3-trifluoromethylphenyl)-3-[1-((E)-1-cyclooct-1-enyl)methyl-piperidin-4-yl]urea

From 4-chloro-3-trifluoromethyl-phenyl isocyanate (17 mg) and 1.0 ml of a 0.075 M solution of Intermediate 3. Yield 9.63 mg. Retention time 2.51 minutes. M+H 444

### EXAMPLE 45

### ′1-(3,5-Bistrifluoromethylphenyl)-3-[1-((E)-1-cyclooct-1-enyl)methyl-piperidin-4-yl]urea

From 3,5-bis(trifluoromethyl)phenyl isocyanate (19 mg) and 1.0 ml of a 0.075 M solution of Intermediate 3. Yield 9.93 mg. Retention time 2.65 minutes. M+H 478

### EXAMPLE 46

### ′1-[1-((E)-1-Cyclooct-1-enyl)methylpiperdin-4-yl]-3-(3-ethylphenyl)urea

From 3-ethylphenyl isocyanate (11 mg) and 1.0 ml of a 0.075 M solution of Intermediate 3. Yield 8.37 mg. Retention time 2.27 minutes. M+H 370

### EXAMPLE 47

### ′1-(3-Chloro-4-methylphenyl)-3-[1-((E)-1-cyclooct-1-enyl)methyl-piperidin-4-yl]urea

From 3-chloro-4-methylphenyl isocyanate (13 mg) and 1.0 ml of a 0.075 M solution of Intermediate 3. Yield 2.23 mg. Retention time 2.35 minutes. M+H 390

### EXAMPLE 48

### 1-(1-Cyclooctylmethyl-piperidin-4-yl)-3-naphthalen-2-yl-urea

Toluene-4-sulfonic acid cyclooctylmethyl ester (CAS No 16472-97-2) (85 mg) and potassium carbonate (120 mg) were added to a solution of Intermediate 8 (85 mg) in anhydrous DMF (5 ml). The reaction mixture was stirred for 17 h at room temperature under a nitrogen atmosphere, then partitioned between water (25 ml) and dichloromethane (25 ml). The organic phase was dried (MgSO₄) and concentrated in vacuo. The residue was triturated with diethyl ether to afford the title compound as a white solid (27 mg). TLC R_{f} 0.42 (10% methanol in dichloromethane). LCMS m/z 394 (MH⁺) observed.

### EXAMPLE 49

### 1-[1-((1R,5S)-6,6-Dimethylbicyclo[3.1.1]hept-2-en-3-ylmethyl)piperidin-4-yl]-3-quinolin-6-yl urea

To a cooled solution (−78° C.) of triphosgene (50 mg) in DCM (1 ml) was added a solution of intermediate 4 (117 mg; 0.5 mmol) in DCM (2.0 ml). After stirring for 1 hour a solution of 6-aminoquinoline (72 mg; 0.5 mmol) and diisopropylethylamine (87 ml) in DCM (1.5 ml) was added and stirring was continued for 18 hours. The reaction mixture was diluted with DCM (50 ml) and washed with aqueous sodium bicarbonate solution (50 ml), dried and evaporated. Purification by preparative HPLC afforded the title compound (55 mg).

Retention time 1.61 minutes. M+H 405

The compound of Example 50 was prepared in a similar manner to the compound of Example 49:

### EXAMPLE 50

### 3-[1-((1R,5S)-6,6-Dimethylbicyclo[3.1.1]hept-2-en-3-ylmethyl)piperidin-4-yl]-1-(3-ethylphenyl)-1-methyl urea

From Intermediate 4 (47 mg; 0.2 mmol) and 3-ethyl-N-methylaniline (27 mg; 0.2 mmol; CAS No. 71265-20-8). Yield 15 mg. Retention time 2.38 minutes. M+H 396

### Biological Assays

The following assays were used to demonstrate the activity and selectivity of compounds according to the invention.

### Chemokine Calcium Assay

The following assay may be used for to determine the inhibition of binding of a chemokine to its receptor:

CHO cells stably transfected with the human CXCR3 were seeded in a 96 well, blackwalled, clear bottomed tissue culture plate and incubated overnight at 37° C. in the presence of 5% CO₂. The culture medium was gently removed from the well and replaced with wash buffer (Hank's Balanced Salts Solution with 0.2% BSA and 20 mM HEPES pH 7.2) containing 3 μM Fluo-4 and 0.03% pluronic acid. The plate was incubated at 37° C. for 1-2 hours, gently washed and 100 μl wash buffer added per well.

Test compounds were dissolved in DMSO and further diluted in wash buffer to give a DMSO concentration of 0.8% (reduced to 0.2% when added to the assay plate in the FLIPR™).

The assay was performed using a FLIPR™ (Molecular Devices). Compound was added to the assay plate after a 10 second baseline. Diluted human recombinant ITAC, IP-10 or MIG was added after a further 2 minutes. Compound activity was calculated as a percentage inhibition of a DMSO solvent control.

Compounds of the invention, for example, the compounds of the Examples, are able to inhibit the binding of ITAC, IP-10 or MIG to their receptor (CXCR3) with an activity of >50% at 5 μm. In this assay the most active compounds according to the invention have IC₅₀ values of around 1 μM or below.

The above assay can also be used to determine the selectivity of the compounds according to the invention, by replacement of CXCR3 with an alternative chemokine receptor such as CCR3 and the use of a chemokine known to bind to such a receptor, such as eotaxin.

In this way the compounds of the invention can be shown to be selective inhibitors of CXCR3. Thus for example the compounds of the Examples are at least 5 times more selective with respect to CXCR3 than to other chemokine receptors such as CCR3.

1. A compound of formula (1): 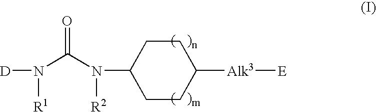
wherein:
m and n, which may be the same or different, are each zero or the integer 1 or 2;
Alk³ is a covalent bond or a straight or branched C₁₋₆ alkylene;
R¹ and R², which may be the same or different, is are each a hydrogen atom or a straight or branched C₁₋₆ alkyl group;
D is an optionally substituted aromatic or heteroaromatic group;
E is an optionally substituted C₇₋₁₀ cycloalkyl, C₇₋₁₀ cycloalkenyl or C₇₋₁₀ polycycloaliphatic group;
and the salts, solvates, hydrates, tautomers or N-oxides thereof.

2. A compound according to claim 1 wherein m and n, which may be the same or different, is are each zero or the integer 1.

3. A compound according to claim 2 wherein m and n are each the integer 1.

4. A compound according to claim 1 wherein Alk³ is a —CH₂—, —CH₂CH₂— or —CH₂CH₂CH₂— chain.

5. A compound according to claim 4 wherein Alk³ is a —CH₂— chain.

6. A compound according to claim 1 wherein R¹ and R², which may be the same or different, are each a hydrogen atom or a methyl group.

7. A compound according to claim 1 wherein D is an optionally substituted phenyl, 1- or 2-naphthyl, pyridyl, pyrimidinyl, pyridazinyl, pyrazinyl, benzofuryl, benzothienyl, indolyl, benzimidazolyl, benzothiazolyl, benzoxazolyl, benzisoxazolyl, quinazolinyl, quinoxalinyl, naphthyridinyl, quinolinyl, isoquinolinyl, pyrrolyl, furyl, thienyl, imidazolyl, N—C₁₋₆alkylimidazolyl, oxazolyl, isoxazolyl, thiazolyl or isothiazolyl group.

8. A compound according to claim 7 wherein D is an optionally substituted phenyl, 2-naphthyl or thienyl group.

9. A compound according to claim 1 wherein E is an optionally substituted cycloheptyl, cyclooctyl, cyclononyl, cyclohexenyl, cycloheptenyl, cyclooctenyl, adamantyl, bicyclo[2.2.1]heptanyl, bicyclo[2.2.1]heptenyl, bicyclo[3.1.1]heptanyl or bicyclo[3.3.1]heptenyl group.

10. A compound according to claim 9 wherein E is a 1-cyclooctenyl, 6,6-dimethylbicyclo[3.1.1]hept-2-en-2-yl, adamantyl or cyclooctyl group.

11. A pharmaceutical composition comprising a compound according to claim 1 together with one or more pharmaceutically acceptable carriers, excipients or diluents.

12. (canceled)

13. A method for the treatment of a disease or disorder in a mammal in which inappropriate T-cell trafficking plays a role, comprising administering to said mammal a therapeutically effective amount of a compound according to claim 1.

14. A method according to claim 13 wherein said disease or disorder is selected from the group consisting of inflammatory or allergic diseases, autoimmune diseases, graft rejection, and diseases in which undesired inflammatory responses are to be inhibited.

15. A method according to claim 14 wherein said disease or disorder is an inflammatory or allergic disease.

16. A method according to claim 15 wherein said inflammatory or allergic disease is selected from the group consisting of systemic anaphylaxis or hypersensitivity responses, drug allergies, insect sting allergies, inflammatory bowel diseases, vaginitis, psoriasis and inflammatory dermatoses, vasculitis, spondyloarthropathies, scleroderma, and respiratory allergic diseases.

17. A method according to claim 14 wherein said disease or disorder is an autoimmune disease.

18. A method according to claim 17 wherein said autoimmune disease is selected from the group consisting of arthritis, multiple sclerosis, systemic lupus erythematosus, diabetes, and glomerulonephritis.

19. A method according to claim 14 wherein said disease or disorder is graft rejection.

20. A method according to claim 14 wherein said disease or disorder is a disease in which an undesired inflammatory response is to be inhibited.

21. A method according to claim 20 wherein said disease is selected from the group consisting of atherosclerosis, myositis, neurodegenerative diseases, Alzheimer's disease, encephalitis, meningitis, hepatitis, nephritis, sepsis, sarcoidosis, conjunctivitis, otitis, chronic obstructive pulmonary disease, sinusitis, Behcet's syndrome, Sjorgen's syndrome and glomerulonephrites.

22. A method for inhibiting, in a mammal suspected to suffer from a disease or disorder in which chemokine-mediated cell signaling plays a role, the binding of chemokines to the CXCR3 receptor, comprising administering to the mammal an effective amount of a compound according to claim 1.
